(19) United States
(12) Patent Application Publication
(10) Pub. No.: US 2004/0167400 A1
(43) Pub. Date: Aug. 26, 2004

(54) Method and apparatus for improving a virtual colonoscopy and A CT angiography

(75) Inventor: Leon Kaufman, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ACCUIMAGE DIAGNOSTICS CORP., South San Francisco, CA (US)

(21) Appl. No.: 10/721,976

(22) Filed: Nov. 25, 2003

Related U.S. Application Data
(60) Provisional application No. 60/429,577, filed on Nov. 27, 2002.

(51) Int. Cl. A61B 6/00

(52) U.S. Cl. 600/431

(57) ABSTRACT

Methods and apparatus for improving a virtual colonoscopy and a CT angiography. Embodiments of the present invention provide a contrast media into the patient's stool or blood stream so as to reduce an attenuation signal of the patient's stool or blood. Reducing the signal of the stool allows an examining physician to better differentiate the stool from polyps in the colon and allows an examining physician to better differentiate the blood from an arterial wall.

### CROSS REFERENCE TO RELATED APPLICATION

The present application claims benefit to U.S. Provisional Patent Application S. No. 60/429,577, filed Nov. 27, 2002, the complete disclosure of which is incorporated herein by reference.

### BACKGROUND OF THE INVENTION

The present invention relates generally to virtual colonoscopies. In particular, the present invention relates to methods of improving a differentiation between a patient's stool and polyps in the colon.

Virtual colonoscopy is one method that allows a physician to image a patient's colon to detect polyps and cancers. Polyps are small growths in the colon that may become cancerous if they are not removed in a timely fashion. Virtual colonoscopy uses a CT scanner and software to image the colon without having to insert a sigmoidoscope or colonoscope (long tube) into the colon.

There are 70 million people in the United States who, by current guidelines, should be screened for colon carcinoma. Conventional screening generally involves three steps: First, the patient needs to be cleaned so that ideally there is no water or stool residue in the colon. Second, the colon is insufflated with typically 1.5-2 liters of air or barium. Third, the viewing device is inserted into the colon and maneuvered to visually examine the colon. If the colon is collapsed or kinked in a certain region, the examination cannot proceed past the obstructed point. The main discomfort to the patient is from the tube (which can also puncture the colon), from the cleaning procedure, and from the insufflation.

Virtual colonoscopy provides an image of the patient's colon that looks similar to an image produced by a conventional colonoscopy. A CT virtual colonoscopy, however, does not use a tube. But, because polyps and the colon wall are indistinguishable by CT numbers, and polyp and feces may also be indistinguishable by CT numbers alone, for the examination of the colon to be successful two things still need to happen: First, the colon has to be cleaned. Second, the colon has to be insufflated. While insufflation is unpleasant, it usually lasts only a short time. Cleaning involves a great deal of discomfort and takes over a day or more to make sure no stool is left in the colon. Even when cleaning, sometimes there is leftover stool. In some instances the stool has some degree of heterogeneity of signal, so that the physician can differentiate between the stool and the polyp. In other instances, however, the stool left in the colon can detrimentally affect the analysis of the colon, since stool is often indistinguishable from polyps by CT numbers alone and the stool may inadvertently be determined to be a polyp.

Investigators are exploring means to differentiate stool from polyp signal in CT by tagging the stool. For example, the patient may feed for a day or two on a diet that includes a tagging element. In radiology there is a bias towards tagging things to appear bright by their increased attenuation of the x-rays. This is a natural inclination in conventional radiography, where obscuring anatomy makes a void harder to see than a hyperintensity, but this is generally of much less of a concern in tomographic imaging.

For example, to differentiate stool from polyps, conventional methods have administered an oral contrast media, such as barium or iodinated compounds so that the barium or iodinated compound mix with the food and enhances the signal of the stool in an attempt to make the stool differentiable from polyps under CT imaging. Unfortunately, the problem with signal enhancement is one of the physics of the imaging device, but the net result is that small polyps next to hyperintensive stool may be missed or their size underestimated. Since size of the polyp is important in guiding treatment, the underestimation of the size of the polyp may have negative consequences.

Consequently, what is needed are apparatus and method for improving a CT virtual colonoscopy.

### BRIEF SUMMARY OF THE INVENTION

The present invention provides methods and apparatus for improving a virtual colonoscopy. Embodiments of the present invention provide a contrast media that reduces an attenuation signal of the patient's stool so as to improve the differentiation between the patient's stool and polyps in the colon. Insufflation is typically still needed because if the colon is collapsed, a polyp may be missed within the folds of the colon wall.

In one embodiment, the methods of the present invention provide orally administering a contrast media to a patient to decrease a signal of stool such that under a CT colonoscopy, the stool will have a lower signal. By lowering the signal of the stool, the examining physician will be able to accurately differentiate polyps from the stool.

In some embodiments, the contrast media can reduce the attenuation signal of the stool to between approximately −100 HU and −200 HU, such that the stool will have a lower attenuation signal than the colon tissue (which is near 0 HU). It should be appreciated however, that other embodiments of the present invention can reduce the attenuation signal of the stool to a higher or lower attenuation signal level, if desired.

In some embodiments, the contrast media can reduce the density of the stool. For example, the contrast media can include hollow polystyrene beads.

In other embodiments, the contrast media can provide an increased fat content to the stool so as to decrease signal intensity.

In other embodiments, the contrast media is a gasogenic food that can stimulate gas formation in the stool so as to decrease its density and increase its heterogeneity. The heterogeneous signal distribution, rather than smooth appearance, provides a visual clue to differentiate a stool from a polyp.

In other embodiments, the contrast media can make the patient's stool more heterogeneous so as to improve the differentiation between the patient's stool and the polyps.

In another aspect, the present invention provides methods and apparatus for improving a CT angiography. Embodiments of the present invention provide a contrast media that reduces an attenuation signal of the patient's blood below an attenuation level of a surrounding arterial wall so as to improve the differentiation between the patient's blood and the arterial walls under computed radiography.

In one embodiment, the present invention provides a method of improving a CT angiography comprising reducing an attenuation signal of a patient's blood to an attenuation level below an attenuation level of a surrounding arterial wall.

In another embodiment, the present invention provides a method of performing a CT angiography. The method comprises administering a contrast media to the patient to reduce an attenuation signal of a patient's blood. Thereafter, the patient's heart is imaged.

In another embodiment, the present invention provides a kit. The kit comprises a contrast media that reduces an attenuation signal of a patient's blood. The kit also includes instructions for use comprising injecting the contrast media a predetermined time prior to performing a CT angiography, wherein the contrast media reduces an attenuation signal of the patient's blood. Optionally, the kit can comprise a package to hold the contrast media and instructions.

Embodiments of the present invention can provide an agent into the bloodstream that decreases the attenuation from the blood so as to provide better visualization of the wall. The agents can be locally injected into the patient's artery with a catheter or needle, ingested orally digested, inhaled, or introduced into the blood stream using any other conventional methods.

Some embodiments of attenuation decreasing agents are fat-laden liposomes and microbubbles. In ultrasound, the surface of the microbubbles may act to increase the echogenecity of the blood. In CT, the microbubbles will reduce the attenuation signal.

For a further understanding of the nature and advantages of the invention, reference should be made to the following description taken in conjunction with the accompanying drawings.

### BRIEF DESCRIPTION OF THE DRAWINGS

FIG. 1 is a point spread function of a CT scanner along one dimension.

FIG. 2 shows the attenuation profile (Object) and resultant detected signal intensity (Signal) along one dimension of a 3-d object that consists of air, a thin region of labeled feces and a start of a polyp, for a scanner with a spatial resolution of 1 mm full-width-half-maximum (FWHM), which is the distance along the half-height of the curve of FIG. 1.

FIG. 3 shows the feces-polyp interface. The scale of FIG. 2 has been amplified in the horizontal and vertical directions so as to better illustrate the effect of interest.

FIG. 4 shows the same information as for FIG. 2 but for feces labeled as per the present invention, so as to reduce its signal intensity.

FIG. 5 is an amplification of the region of interest of FIG. 4.

### DETAILED DESCRIPTION OF THE INVENTION

Embodiments of the present invention improve a virtual colonoscopy by reducing a signal of the stool through an addition of a contrast media to the patient's stool. The present invention further improve a CT angiography by administering a contrast media to the patient to reduce an attenuation signal of a patient's blood.

Embodiments of the present invention can use an orally ingested contrast media to reduce the CT signal of the stool (and/or blood). Physically, if the patient's stool has a high attenuation signal, as is created when barium is ingested, the stool creates a zone around it where the tissue attenuation signal, including the attenuation signal generated by polyps in the colon, is lost. As a consequence, when the signal from the stool is eliminated through thresholding, the colon wall often becomes artifactually thinned, and polyps are artifactually reduced in diameter. An artifact is a feature in an image that does not represent the reality of the object, a deviation from reality. Since the criteria for polyp removal are typically based on polyp diameter, thinning of the diameter of the polyps can have serious consequences on the type of patient treatment.

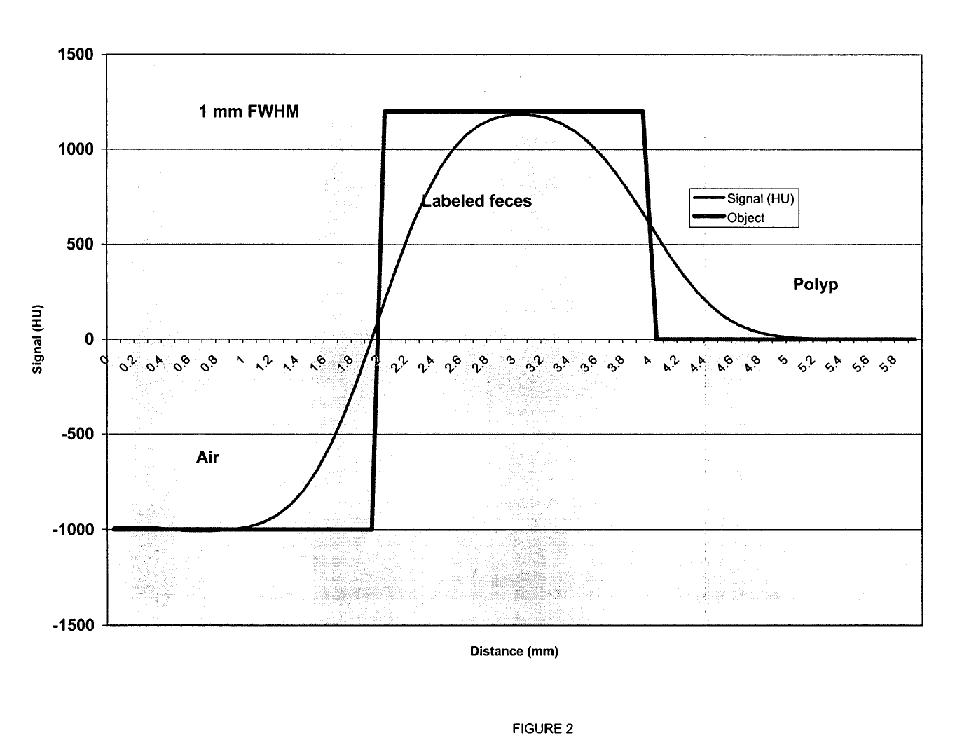

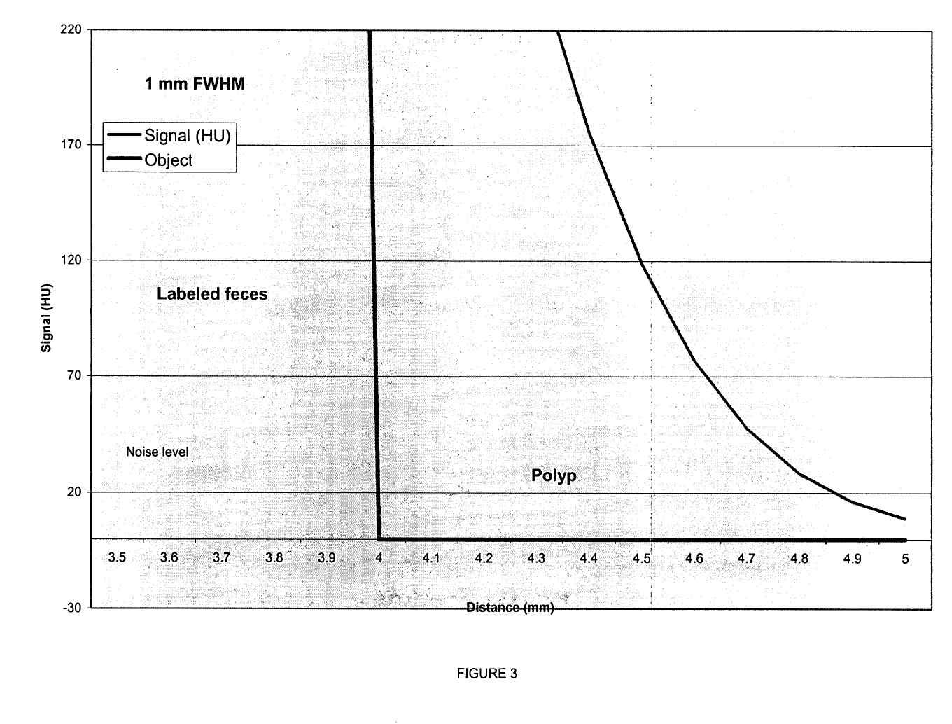

Some embodiments of the present invention reduce the attenuation signal of the stool to below that of the surrounding tissue (which is roughly the same as the signal from water). As can be appreciated from FIGS. 2 and 3, when the object is hyperintense its signal “spills” into the region occupied by the polyp. To differentiate polyp from feces, some threshold for the signal level has to be set. This threshold has to be large enough to exclude noise, but not so large that the polyp signal is distorted. For instance, in conventional modern CT scanners, a signal of 90 HU is considered a calcification, and the noise may be 20 HU or less. As can be appreciated from FIG. 3, a threshold between approximately 20 HU and 90 HU will result in a “shaving off” of approximately 0.6 mm-0.9 mm from the edge of the polyp, or a total of 1.2 to 1.8 mm for a rounded polyp. Thus, a 6 mm polyp (which according to some guidelines should be extirpated), will seem to have less than 5 mm, which by the same guidelines would be left without intervention.

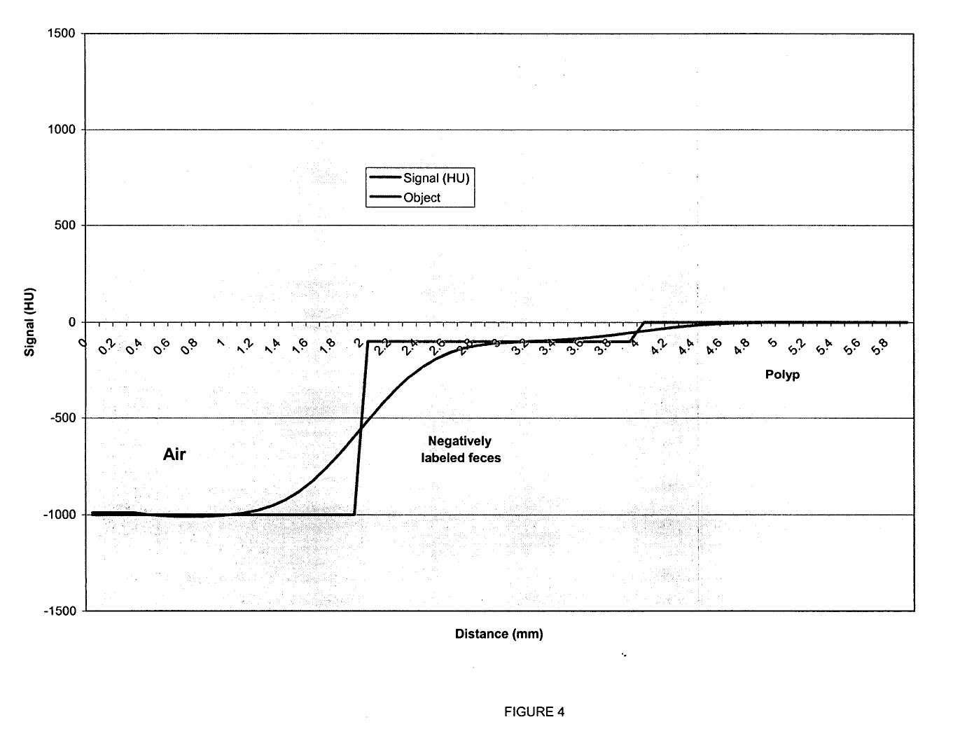

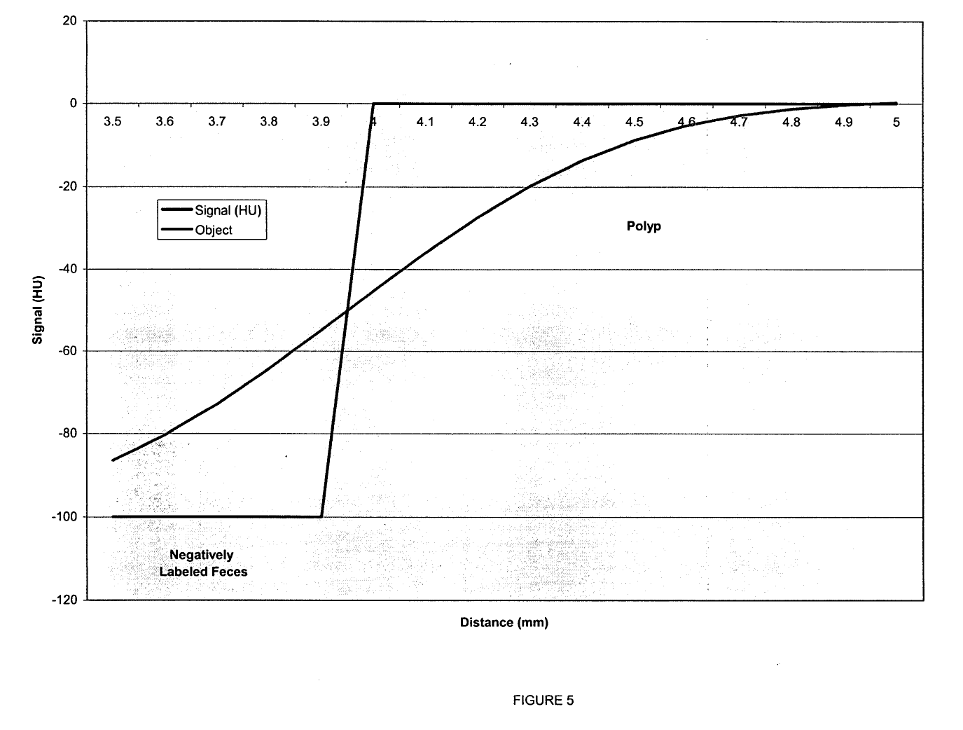

In the embodiments of the present invention, when the signal from feces is reduced to less than the signal from the polyps, as in FIGS. 4 and 5, a threshold of −20 HU to −50 HU will result in no more than approximately a 0.3 mm “shaving”, for a total apparent reduction of less than approximately 0.6 mm in the size of the polyp.

Embodiments of the contrast media of the present invention can comprise materials of low density that can be added to the patient's diet. One example of a contrast media is cellulose, e.g., sawdust, which is added to diet breads to create bulk while not delivering calories. Cellulose has lower density than water and will contribute to reducing the density of stool. Another example of the contrast media is hollow polystyrene beads, which are of very low density and are not digested.

In other embodiments, the contrast media can provide an increased fat content to the stool so as to decrease signal intensity.

In other embodiments, the contrast media is a gasogenic food that can stimulate gas formation in the stool. For example, the contrast media may produce gas bubbles. Such gas bubbles will reduce the density of the stool.

In other embodiments, the contrast media can make the patient's stool more heterogeneous so as to improve the differentiation between the patient's stool and the polyps.

The gasogenic contrast media can be released into the stool in a variety of methods. In one embodiment, release of the contrast media is initiated by a chemical found in the colon. In other embodiments, release of the contrast media can be controlled by the time it takes to reach the colon (the principle behind time release capsules for various medications).

In other embodiments, the release of the contrast media can be initiated by a separately administered chemical that could itself be in a time release capsule. In other embodiments, the release of the contrast media can be initiated by an external stimulus. For example, there are intravascular ultrasound contrast media that release microbubbles when irradiated by an ultrasound beam. If the contrast media were coated so as to pass through the stomach, the bubbles could be released by delivery of an ultrasound beam to the patients prior to the CT virtual colonoscopy.

Embodiments of the present invention also provide software and apparatus for improving the accuracy of a virtual colonoscopy. Prior to performing the virtual colonoscopy, the physician can instruct the patient to take ingest the contrast media a predetermined amount of time (typically 1 to 2 days) prior to the virtual colonoscopy. The contrast media can be in the form of a pill, a suspension, a liquid mixed, baked or cooked into a particular food, a combination of these, or the like.

An image scanner, such as a CT scanner, a 3D CT scanner, ultrasound, or MRI scanner, can be used to scan the patient's abdomen. During such an image scan, a series of two-dimensional slices of the patient's colon will be obtained. The slices of the patient's colon can also be reconstructed using conventional methods in the art to generate a three-dimensional representation of the patient's colon. The slices of the patient's colon can then be thresholded to as to isolate the colon from the air column and stool that remains in the patient's colon. In exemplary methods, embodiments of the software of the present invention can apply an attenuation threshold that is below the attenuation level of the colon, typically between −20 HU and −50 HU, or lower. Such a threshold separates the colon (and polyps) from the air column and stool and improves the analysis of the colon for polyps.

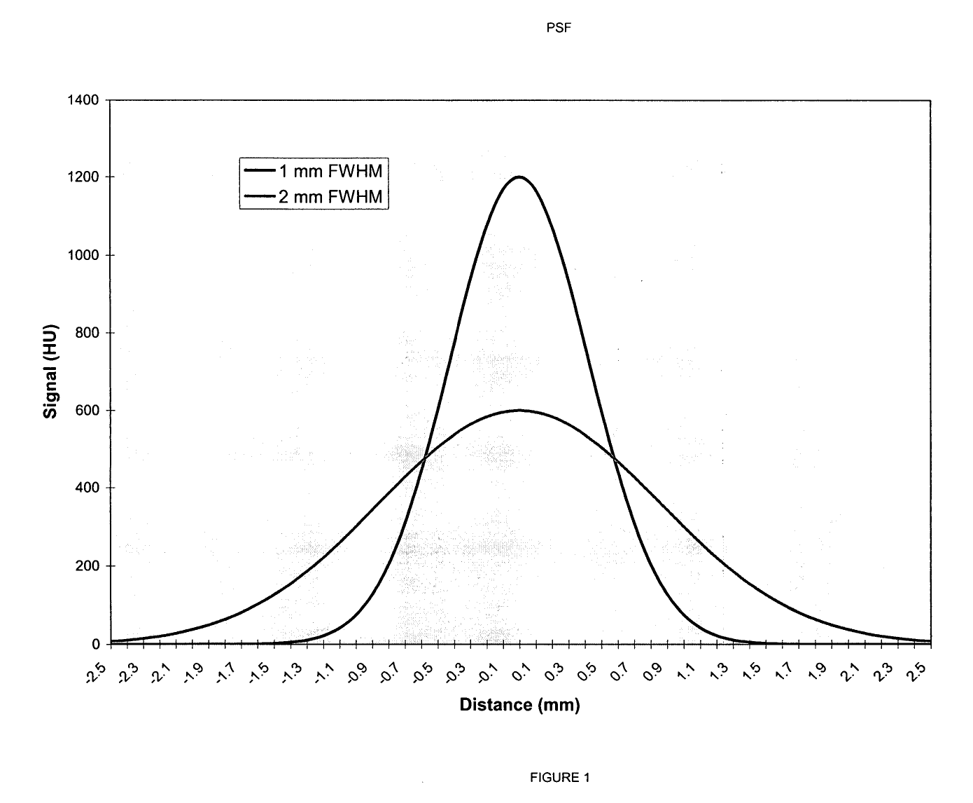

Referring again to FIGS. 1-3. FIG. 1, represents a point spread function for a CT scanner along one dimension. The scanner spreads the signal of an infinitesimally small point over a finite space. Any object can be thought of as being composed of a large number of such points, so that the signal from the object can be modeled on this basis. The scanner also spreads the signal along the slice dimension, which traditionally has been larger than the in-plane resolution. Modern scanners can produce nearly isotropic resolution images, even though the profile of the resolution function along the slice direction differs in detail from that of FIG. 1. The range of FWHMs of commercial CT scanners is generally 1 mm to 2 mm. This example is for 1 mm resolution. For 2 mm the effect is twice as large.

FIG. 2 shows along one dimension of a 3-D object that is comprised of air, a thin region of tagged feces, and then the start of a polyp that extends farther to the right. is the area of concern is what happens at the interface between feces and polyp. FIG. 3 focuses on the interface between the feces and polyp. As can be seen in FIG. 3, the signal from the feces extends into the polyp. If a user sets the threshold above noise, say, 20 HU, the edge of the polyp as been decreased by about one FWHM, in this case, by approximately 0.9 mm for 1 mm FWHM. Even for a high threshold, say, 120 HU (already a level that would be called a calcification), the edge is reduced by 0.5 mm. In this manner, even for a 1 mm FWHM, the size of a rounded polyp may be reduced between approximately 1 mm and 2 mm, a difference that may make a difference on whether it is surgically removed or not.

While all the above is a complete description of the preferred embodiments of the inventions, various alternatives, modifications, and equivalents may be used. For example, in CT angiography, an iodinated or gadolinium-based contrast agent is given to distinguish the lumen of the vessel from its surroundings. Both of these contrast agents increase attenuation in the blood, i.e., yield a hyperintense signal. It is now believed that the coronary lesions that rupture, leading to heart attacks, may not intrude into the lumen to a significant extent, and there is interest in locating these lesions non-invasively. This means obtaining data from the wall of the vessel, which is in intimate contact with the contrast agent-laden blood in the lumen. The same effect that leads to apparent polyp size reduction will obscure the edge of the wall, making it difficult to obtain adequate information. If instead of an agent that increases the attenuation from the blood there was administration of an agent that decreases it, this effect would be ameliorated and better visualization of the wall would be obtained. Candidate agents are fat-laden liposomes and microbubbles. The latter have been used in ultrasound imaging, where the surface of the bubbles act to increase the echogenecity of the blood. In CT they will reduce the attenuation signal. Although the foregoing invention has been described in detail for purposes of clarity of understanding, it will be obvious that certain modifications may be practiced within the scope of the appended claims.

What is claimed is:

1. A method of improving a virtual colonoscopy comprising reducing an attenuation signal of a patient's stool to an attenuation level below an attenuation level of a surrounding colon tissue.

2. The method of claim 1 wherein reducing the signal comprises introducing a contrast media in the patient's stool.

3. The method of claim 2 wherein introducing the contrast media comprises orally ingesting the contrast media.

4. The method of claim 2 wherein the contrast media lowers the density of the patient's stool.

5. The method of claim 4 wherein the contrast media reduces the patient's stool to a density between 80% and 90% that of colon tissue.

6. The method of claim 4 wherein the contrast media is polystyrene hollow beads.

7. The method of claim 4 wherein the contrast media comprises cellulose.

8. The method of claim 7 wherein the contrast media comprises a preparation derived from sawdust.

9. The method of claim 2 wherein the contrast media stimulates gas formation in the patient's stool.

10. The method of claim 9 wherein the contrast media comprises gasogens.

11. The method of claim 10 wherein the contrast media comprises air inside a membrane.

12. The method of claim 11 wherein the contrast media comprises an air filled closed cell foam.

13. The method of claim 13 wherein the contrast media reduces the patient's stool to an attenuation signal between approximately −100 HU and −200 HU.

14. The method of claim 2 wherein the contrast media makes the patient's stool more heterogeneous.

15. The method of claim 2 wherein the contrast media increases a fat content of the patient's stool.

16. A method of performing a virtual colonoscopy, the method comprising:
administering a contrast media to the patient to reduce an attenuation signal of a patient's stool; and
imaging the patient's colon.

17. The method of claim 16 wherein administering a contrast media comprises orally ingesting the contrast media.

18. The method of claim 16 wherein the contrast media lowers a density of the patient's stool.

19. The method of claim 18 wherein the contrast media lowers the density of the patient's stool to between approximately 80% and 90% that of colon tissue.

20. The method of claim 18 wherein the contrast media is polystyrene foam beads.

21. The method of claim 18 wherein the contrast media comprises cellulose.

22. The method of claim 21 wherein the contrast media comprises a product derived from sawdust.

23. The method of claim 16 wherein the contrast media stimulates gas formation in the patient's stool.

24. The method of claim 23 wherein the contrast media comprises gasogens.

25. The method of claim 24 wherein the contrast media comprises air inside a membrane.

26. The method of claim 25 wherein the contrast media comprises an air filled closed cell foam.

27. The method of claim 16 wherein the contrast media reduces the patient's stool to an attenuation signal between approximately −100 HU and −200 HU.

28. The method of claim 16 wherein the contrast media makes the patient's stool more heterogeneous.

29. The method of claim 16 wherein the contrast media increases a fat content of the patient's stool.

30. A kit for improving a virtual colonoscopy, the kit comprising:
a contrast media that reduces an attenuation signal of a patient's stool;
instructions for use comprising orally ingesting the contrast media a predetermined time prior to performing a virtual colonoscopy, wherein the contrast media reduces an attenuation signal of the patient's stool; and
a package to hold the contrast media and instructions.

31. The kit of claim 30 wherein the contrast media reduces a density of the patient's stool.

32. The kit of claim 30 wherein the contrast media increases a fat content in the patient's stool.

33. The kit of claim 30 wherein the contrast media makes the patient's stool more heterogeneous.

34. The kit of claim 30 wherein the contrast media stimulates gas formation in the patient' stool.

35. A method of performing a virtual colonoscopy on a computer system, the method comprising:
accessing an image scan of a patient's colon; and
applying an attenuation threshold that has a lower threshold than an attenuation signal of the patient's colon so as to isolate a patient's stool from the patient's colon.

36. A method of improving a CT angiography comprising reducing an attenuation signal of a patient's blood to an attenuation level below an attenuation level of a surrounding arterial wall.

37. The method of claim 36 wherein reducing the signal comprises introducing a contrast media in the patient's blood by means of an injection.

38. The method of claim 36 wherein the contrast media lowers the density of the patient's blood.

39. The method of claim 38 wherein the contrast media is microscopic air bubbles.

40. The method of claim 38 wherein the contrast media comprises fat-laden liposomes.

41. A method of performing a CT angiography, the method comprising:
administering a contrast media to the patient to reduce an attenuation signal of a patient's blood; and
imaging the patient's heart.

42. The method of claim 41 wherein the contrast media lowers a density of the patient's blood.

43. The method of claim 42 wherein the contrast media is microscopic air bubbles.

44. The method of claim 42 wherein the contrast media comprises fat-laden liposomes.

45. A kit for improving a CT angiography, the kit comprising:
a contrast media that reduces an attenuation signal of a patient's blood;
instructions for use comprising injecting the contrast media a predetermined time prior to performing a CT angiography, wherein the contrast media reduces an attenuation signal of the patient's blood; and
a package to hold the contrast media and instructions.
